(19) United States
(12) Patent Application Publication
(10) Pub. No.: US 2012/0022078 A1
(43) Pub. Date: Jan. 26, 2012

(54) ARYL INDOLE DERIVATIVES

(75) Inventors: Hiroyuki Kishino, Edogawa-ku (JP); Sayaka Mizutani, Tsuchiura-shi (JP); Shunji Sakuraba, Tsukuba-shi (JP); Nagaaki Sato, Saitama-shi (JP)

(73) Assignee: MSD K.K., Chiyoda-ku (JP)

(21) Appl. No.: 13/258,006

(22) PCT Filed: Apr. 7, 2010
(86) PCT No.: PCT/JP2010/056636

§ 371 Date: Oct. 18, 2011

(51) Int. Cl. A61K 31/496; A61P 3/04; C07D 403/10; C07D 417/14; C07D 401/14

(52) U.S. Cl. 514/253.09; 544/369; 514/254.02; 544/364; 544/373; 514/254.09

(57) ABSTRACT

A novel aryl indole derivative is provided that is effective as a preventive or remedy for various diseases. A compound represented by a formula (I) or a pharmaceutically acceptable salt thereof: (I) wherein R¹ is a hydrogen atom, halogen, C₁₋₆alkyl, haloC₁₋₆alkyl, C₁₋₆alkyloxy, or haloC₁₋₆alkyloxy; R² represents C₁₋₆alkyl or haloC₁₋₆alkyl; R³ represents a hydrogen atom, C₁₋₆alkyl, or haloC₁₋₆alkyl; and Ar represents an aryl or heteroaryl, wherein the aryl or the heteroaryl may be substituted with 1 to 3 substituents such as halogen, C₁₋₆alkyl, haloC₁₋₆alkyl, and C₁₋₆alkyloxy.

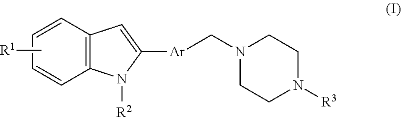

### TECHNICAL FIELD

The present invention relates to aryl indole derivatives useful as medicaments. The compounds act as human QRFP receptor (GPR103) antagonists, and are useful as preventive or remedy for obesity.

### BACKGROUND ART

QRFP43, a peptide of 43 amino acids, has been reported as an endogenous ligand of QRFP43 receptor (GPR103) through bioinformatics and reverse pharmacology (2003), and it was first isolated from rat brains in 2006 (see, for example, Non-Patent Document 1). There are also reports that, for example, 26RFa, a close relative of QRFP43, binds to QRFP43 receptor, and has activity similar to that of QRFP43 (see, for example, Patent Documents 1 and 2). QRFP43 is expressed at high level in the central nervous system, particularly in the hypothalamus, and has a diversity of functions in the body. Specifically, QRFP43 acts as a centrally-acting appetite promoter, and promotes prominent fat accumulation through secretion of various hormones or actions of the nervous system. It is known that intracerebroventricular successive administration of QRFP43 induces obesity and insulin resistance based on these actions. QRFP43 is also involved in hormone secretion such as in the hypothalamus and pituitary gland.

The functions of QRFP43 or 26RFa are expressed upon binding to the QRFP43 receptor (GPR103) present in the central or peripheral nervous system. It would therefore be possible to inhibit the functional expression of QRFP43 or 26RFa by inhibiting the binding of QRFP43 or 26RFa to the QRFP43 receptor (GPR103).

Compounds relating to aryl indole derivatives of the present invention can be found in, for example, Patent Document 3. However, the compounds described in this publication have structures that require an amino group at position 7 of the indole, and therefore differ from the compounds of the present invention which do not have an amino group at position 7 of the indole. Further, the compounds of the foregoing publication are cell necrosis factor inhibitors, and are not described as the QRFP43 receptor antagonists as in the present invention. Further, to date, there has been no report of low molecular QRFP43 receptor antagonists.

Non-Patent Document 1: Proceedings of the National Academy of Sciences of the United States of America, Vol. 103, pp. 7438-7443, (2006)

Patent Document 1: WO001/16316

Patent Document 2: WO005/65702

Patent Document 3: WO009/25478

### DISCLOSURE OF THE INVENTION

### Problems that the Invention is to Solve

Because the inhibition of the QRFP43 or 26RFa binding to the QRFP43 receptor (GPR103) can inhibit the functional expression of QRFP43 or 26RFa, a substance antagonistic to the QRFP43 binding to the QRFP43 receptor (GPR103) is expected to be useful in the prevention or treatment of various diseases involving QRFP43 or 26RFa, including, for example, cardiovascular disease such as hypertension, arterial sclerosis, renal disease, heart disease, and angiospasm; bulimia; and metabolic disease such as obesity, diabetes mellitus, abnormal hormone secretion, hypercholesteremia, hyperlipidemia, gout, and fatty liver. Such a substance can therefore be provided as a preventive or remedy for, for example, pain, abnormal circadian rhythms, atherosclerosis, obesity-related gastroesophageal reflux, obesity-hypoventilation syndrome (Pickwickian syndrome), hypertriglyceridemia, low HDL cholesteremia, cardiovascular disease (for example, such as coronary artery heart disease (CHD), cerebrovascular disease, stroke, peripheral vascular disease, and sudden death), pain, osteoporosis-related disease, lower back pain, and anesthetic hypersensitivity.

It is accordingly an object of the present invention to provide an antagonist for QRFP43 receptor (GPR103), which is useful as a preventive or remedy for diseases such as above.

### Means for Solving the Problems

After intensive studies, the inventors of the present invention have found that compounds including a benzene ring or a specific heteroaryl ring substituting at position 2 of an indole skeleton have an excellent human QRFP receptor (GPR103) antagonist activity. The present invention was completed based on this finding.

Specifically, the present invention provides,

(1) A compound represented by a formula (I) or a pharmaceutically acceptable salt thereof:

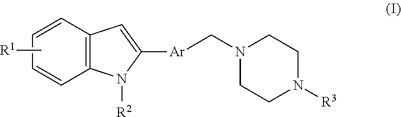

wherein R¹ represents a hydrogen atom, halogen, C₁₋₆alkyl, haloC₁₋₆alkyl, C₁₋₆alkyloxy, or haloC₁₋₆alkyloxy;

R² represents C₁₋₆alkyl or haloC₁₋₆alkyl;

R³ represents a hydrogen atom, C₁₋₆alkyl, or haloC₁₋₆alkyl; and

Ar represents an aryl or heteroaryl, wherein the aryl or the heteroaryl may be substituted with 1 to 3 substituents selected from the group consisting of halogen, C₁₋₆alkyl, haloC₁₋₆alkyl, C₁₋₆alkyloxy, haloC₁₋₆alkyloxy, hydroxy, amino, monoC₁₋₆alkylamino, diC₁₋₆alkylamino, C₁₋₆alkyloxycarbonyl, C₁₋₆alkyloxycarbonylamino, C₁₋₆alkyloxycarbonyl(C₁₋₆alkyl)amino, C₁₋₆alkylcarbonyl, C₁₋₆alkylcarbonyloxy, C₁₋₆alkylcarbonylamino, C₁₋₆alkylcarbonyl(C₁₋₆alkyl)amino, carbamoyl, monoC₁₋₆alkylcarbamoyl, diC₁₋₆alkylcarbamoyl, carbamoylamino, monoC₁₋₆alkylcarbamoylamino, diC₁₋₆alkylcarbamoylamino, monoC₁₋₆alkylcarbamoyl(C₁₋₆alkyl)amino, diC₁₋₆alkylcarbamoyl(C₁₋₆alkyl)amino, carbamoyloxy, monoC₁₋₆alkylcarbamoyloxy, diC₁₋₆alkylcarbamoyloxy, C₁₋₆alkylsulfonyl, C₁₋₆alkylsulfonylamino, C₁₋₆alkylsulfonyl(C₁₋₆alkyl)amino, sulfamoyl, monoC₁₋₆alkylsulfamoyl, diC₁₋₆alkylsulfamoyl, sulfamoylamino, monoC₁₋₆alkylsulfamoylamino, diC₁₋₆alkylsulfamoylamino, monoC₁₋₆alkylsulfamoyl(C₁₋₆alkyl)amino, and diC₁₋₆alkylsulfamoyl(C₁₋₆alkyl)amino,

(2) A pharmaceutical composition which contains a compound of (1) or a pharmaceutically acceptable salt thereof, and a pharmaceutically acceptable carrier, and

(3) A remedy for obesity, which contains a compound of (1) or a pharmaceutically acceptable salt thereof as an active ingredient.

### BEST MODE FOR CARRYING OUT THE INVENTION

The present invention is described below in more detail.

Examples of the “halogen” include fluoro, chloro, bromo, and iodo.

The “C₁₋₆alkyl” includes straight-chain alkyls having 1 to 6 carbon atoms, and branched alkyls having 3 to 6 carbon atoms. Specific examples include methyl, ethyl, n-propyl, isopropyl, n-butyl, isobutyl, sec-butyl, tert-butyl, n-pentyl, isopentyl, neopentyl, tert-amyl, 2-propyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, and 1-ethyl-1-methylpropyl.

The “haloC₁₋₆alkyl” includes C₁₋₆alkyls in which some of or all of the hydrogen atoms are substituted with halogens. Examples include fluoromethyl, difluoromethyl, trifluoromethyl, 2-fluoroethyl, and 1,2-difluoroethyl.

The “C₁₋₆alkyloxy” includes groups with the C₁₋₆alkyl attached to an oxygen atom. Specific examples include methoxy, ethoxy, n-propyloxy, isopropyloxy, n-butyloxy, isobutyloxy, tert-butyloxy, and n-pentyloxy.

The “haloC₁₋₆alkyloxy” includes groups with the haloC₁₋₆alkyl attached to an oxygen atom. Specific examples include fluoromethoxy, chloromethoxy, difluoromethoxy, dichloromethoxy, trifluoromethoxy, trichloromethoxy, 2-fluoroethoxy, and 1,2-difluoroethoxy.

The “monoC₁₋₆alkylamino” is a group in which one of the hydrogen atoms of the amino (—NH₂) is substituted with a C₁₋₆alkyl group. Specific examples include methylamino, ethylamino, n-propylamino, isopropylamino, n-butylamino, sec-butylamino, and tert-butylamino.

The “diC₁₋₆alkylamino” is a group in which the two hydrogen atoms of the amino are substituted with C₁₋₆alkyls. Specific examples include dimethylamino, diethylamino, ethylmethylamino, di(n-propyl)amino, methyl(n-propyl)amino, and diisopropylamino.

The “C₁₋₆alkyloxycarbonyl” is a group with the C₁₋₆alkyloxy attached to a carbonyl (—CO—), and includes alkyloxycarbonyls having 1 to 6 carbon atoms. Specific examples include methoxycarbonyl, ethoxycarbonyl, n-propyloxycarbonyl, isopropyloxycarbonyl, n-butoxycarbonyl, isobutoxycarbonyl, tert-butoxycarbonyl, and n-pentyloxycarbonyl.

The “C₁₋₆alkyloxycarbonylamino” is a group in which one of the hydrogen atoms of the amino is substituted with a C₁₋₆alkyloxycarbonyl, and includes alkyloxycarbonylaminos having 1 to 6 carbon atoms. Specific examples include methoxycarbonylamino, ethoxycarbonylamino, n-propyloxycarbonylamino, isopropyloxycarbonylamino, n-butoxycarbonylamino, isobutoxycarbonylamino, tert-butoxycarbonylamino, and n-pentyloxycarbonylamino.

The “C₁₋₆alkyloxycarbonyl(C₁₋₆alkyl)amino” is a group in which a C₁₋₆alkyloxycarbonyl is attached in place of the hydrogen atom on the nitrogen atom of the monoC₁₋₆alkylamino. Specific examples include methoxycarbonyl(methyl)amino, ethoxycarbonyl(methyl)amino, and n-propyloxycarbonyl(methyl)amino.

The “C₁₋₆alkylcarbonyl” is a group with the C₁₋₆alkyl attached to a carbonyl, and includes alkylcarbonyls having 1 to 6 carbon atoms. Specific examples include acetyl, propionyl, butyryl, isobutyryl, valeryl, isovaleryl, and pivaloyl.

The “C₁₋₆alkylcarbonyloxy” is a group with the C₁₋₆alkylcarbonyl attached to an oxygen atom. Specific examples include acetoxy, propionyloxy, valeryloxy, isovaleryloxy, and pivaloyloxy.

The “C₁₋₆alkylcarbonylamino” is a group in which one of the hydrogen atoms of the amino group is substituted with a C₁₋₆alkylcarbonyl. Specific examples include acetylamino, propionylamino, isobutyrylamino, valerylamino, isovalerylamino, and pivaloylamino.

The “C₁₋₆alkylcarbonyl(C₁₋₆alkyl)amino” is a group in which the hydrogen atom on the nitrogen atom of the monoC₁₋₆alkylamino is substituted with a C₁₋₆alkylcarbonyl. Examples include methylcarbonyl(methyl)amino, ethylcarbonyl(methyl)amino, and n-propylcarbonyl(methyl)amino.

The “monoC₁₋₆alkylcarbamoyl” is a group in which one of the hydrogen atoms of the carbamoyl (—CONH₂) is substituted with a C₁₋₆alkyl. Specific examples include methylcarbamoyl, ethylcarbamoyl, n-propylcarbamoyl, isopropylcarbamoyl, n-butylcarbamoyl, sec-butylcarbamoyl, and tert-butylcarbamoyl.

The “diC₁₋₆alkylcarbamoyl” is a group in which the two hydrogen atoms of the carbamoyl are substituted with C₁₋₆alkyls. Specific examples include dimethylcarbamoyl, diethylcarbamoyl, ethylmethylcarbamoyl, di(n-propyl)carbamoyl, methyl(n-propyl)carbamoyl, and diisopropylcarbamoyl.

The “monoC₁₋ₒalkylcarbamoylamino” is a group in which one of the hydrogen atoms of the amino is substituted with a monoC₁₋₆alkylcarbamoyl. Specific examples include methylcarbamoylamino, ethylcarbamoylamino, n-propylcarbamoylamino, isopropylcarbamoylamino, n-butylcarbamoylamino, sec-butylcarbamoylamino, and tert-butylcarbamoylamino.

The “diC₁₋₆alkylcarbamoylamino” is a group in which one of the hydrogen atoms of the amino is substituted with a diC₁₋₆alkylcarbamoyl. Specific examples include dimethylcarbamoylamino, diethylcarbamoylamino, di(n-propyl)carbamoylamino, diisopropylcarbamoylamino, di(n-butyl)carbamoylamino, di(sec-butyl)carbamoylamino, and di(tert-butyl)carbamoylamino.

The “monoC₁₋₆alkylcarbamoyl(C₁₋₆alkyl)amino” is a group in which the hydrogen atom on the nitrogen atom of the monoC₁₋₆alkylamino is substituted with a monoC₁₋₆alkylcarbamoyl. Specific examples include monomethylcarbamoyl(methyl)amino, monoethylcarbamoyl(methyl)amino, and [mono(n-propyl)carbamoyl](methyl)amino.

The “diC₁₋₆alkylcarbamoyl(C₁₋₆alkyl)amino” is a group in which the hydrogen atom on the nitrogen atom of the monoC₁₋₆alkylamino is substituted with a diC₁₋₆alkylcarbamoyl. Specific examples include dimethylcarbamoyl(methyl)amino, diethylcarbamoyl(methyl)amino, and [di(n-propyl)carbamoyl](methyl)amino.

The “monoC₁₋₆alkylcarbamoyloxy” is a group with the monoC₁₋₆alkylcarbamoyl attached to an oxygen atom. Specific examples include methylcarbamoyloxy, ethylcarbamoyloxy, n-propylcarbamoyloxy, isopropylcarbamoyloxy, n-butylcarbamoyloxy, sec-butylcarbamoyloxy, and tert-butylcarbamoyloxy.

The “diC₁₋₆alkylcarbamoyloxy” is a group with the diC₁₋₆alkylcarbamoyl attached to an oxygen atom. Specific examples include dimethylcarbamoyloxy, diethylcarbamoyloxy, ethylmethylcarbamoyloxy, di(n-propyl)carbamoyloxy, methyl(n-propyl)carbamoyloxy, and diisopropylcarbamoyloxy.

The “C₁₋₆alkylsulfonyl” is a group with the C₁₋₆alkyl attached to a sulfonyl (—SO₂—).

Specific examples include methanesulfonyl, ethanesulfonyl, n-propanesulfonyl, isopropanesulfonyl, n-butanesulfonyl, sec-butanesulfonyl, and tert-butanesulfonyl.

The “C₁₋₆alkylsulfonylamino” is a group in which one of the hydrogen atoms of the amino is substituted with a C₁₋₆alkylsulfonyl. Specific examples include methanesulfonylamino, ethanesulfonylamino, n-propanesulfonylamino, isopropanesulfonylamino, n-butanesulfonylamino, sec-butanesulfonylamino, and tert-butanesulfonylamino.

The “C₁₋₆alkylsulfonyl(C₁₋₆alkyl)amino” is a group in which the hydrogen atom on the nitrogen atom of the monoC₁₋₆alkylamino is substituted with a C₁₋₆alkylsulfonyl. Specific examples include methanesulfonyl(methyl)amino, ethanesulfonyl(methyl)amino, n-propanesulfonyl(methyl)amino, and isopropanesulfonyl(methyl)amino.

The “monoC₁₋₆alkylsulfamoyl” is a group in which one of the hydrogen atoms of the sulfamoyl(—SO₂NH₂) is substituted with a C₁₋₆alkyl. Specific examples include monomethylsulfamoyl, monoethylsulfamoyl, mono(n-propyl)sulfamoyl, monoisopropylsulfamoyl, mono(n-butyl)sulfamoyl, mono(sec-butyl)sulfamoyl, and mono(tert-butyl)sulfamoyl.

The “diC₁₋₆alkylsulfamoyl” is a group in which the two hydrogen atoms of the sulfamoyl are substituted with C₁₋₆alkyls. Specific examples include dimethylsulfamoyl, diethylsulfamoyl, di(n-propyl)sulfamoyl, diisopropylsulfamoyl, di(n-butyl)sulfamoyl, di(sec-butyl)sulfamoyl, and di(tert-butyl)sulfamoyl.

The “monoC₁₋₆alkylsulfamoylamino” is a group in which one of the hydrogen atoms of the amino is substituted with a monoC₁₋₆alkylsulfamoyl. Specific examples include (monomethylsulfamoyl)amino, (monoethylsulfamoyl)amino, [mono(n-propyl)sulfamoyl]amino, (monoisopropylsulfamoyl)amino, [mono(n-butyl)sulfamoyl]amino, [mono(sec-butyl)sulfamoyl]amino, and [mono(tert-butyl)sulfamoyl]amino.

The “(diC₁₋₆alkylsulfamoyl)amino” is a group in which one of the hydrogen atoms of the amino is substituted with a diC₁₋₆alkylsulfamoyl. Specific examples include (dimethylsulfamoyl)amino, (diethylsulfamoyl)amino, (ethylmethylsulfamoyl)amino, [di(n-propyl)sulfamoyl]amino, [methyl(n-propyl)sulfamoyl]amino, and (diisopropylsulfamoyl)amino.

The “monoC₁₋₆alkylsulfamoyl(C₁₆alkyl)amino” is a group in which the hydrogen atom on the nitrogen atom of the monoC₁₋₆alkylamino is substituted with a monoC₁₋₆alkylsulfamoyl. Specific examples include monomethylsulfamoyl(methyl)amino, monoethylsulfamoyl(methyl)amino, and [mono(n-propyl)sulfamoyl](methyl)amino.

The “diC₁₋₆alkylsulfamoyl(C₁₋₆alkyl)amino” is a group in which the hydrogen atom on the nitrogen atom of the monoC₁₋₆alkylamino is substituted with a diC₁₋₆alkylsulfamoyl. Specific examples include dimethylsulfamoyl(methyl)amino, diethylsulfamoyl(methyl)amino, and [di(n-propyl)sulfamoyl](methyl)amino.

Examples of the “aryl” include phenyl and naphthyl.

The “heteroaryl” means a five- or six-membered monocyclic heteroaryl having one or more, preferably 1 to 2 heteroatoms, the same or different, selected from the group consisting of oxygen atom, nitrogen atom, and sulfur atom, or a condensed-ring heteroaryl formed by the condensation of the monocyclic heteroaryl and the aryl, or by the condensation of the monocyclic heteroaryls which may be the same or different. Examples include pyrrolyl, furyl, thienyl, imidazolyl, pyrazolyl, thiazolyl, isothiazolyl, oxazolyl, isooxazolyl, triazolyl, tetrazolyl, oxadiazolyl, 1,2,3-thiadiazolyl, 1,2,4-thiadiazolyl, 1,3,4-thiadiazolyl, pyridyl, pyrazinyl, pyrimidinyl, pyridazinyl, 1,2,4-triazinyl, 1,3,5-triazinyl, indolyl, benzofuranyl, benzothienyl, benzimidazolyl, benzopyrazolyl, benzoxazolyl, benzisooxazolyl, benzthiazolyl, benzisothiazolyl, indazolyl, purinyl, quinolyl, isoquinolyl, phthalazinyl, naphthyridinyl, quinoxalinyl, quinazolinyl, cinnolinyl, pteridinyl, and pyrido[3,2-b]pyridyl.

The “pharmaceutically acceptable salt” of a derivative represented by the formula (I) includes medicinally acceptable salts commonly used. Examples include an acid addition salt formed at the amine moiety of the compound of the formula (I), or an acid addition salt formed at the nitrogen-containing heterocyclic ring, and, when the compound of the formula (I) has an acidic substituent, a base addition salt formed at such a group.

Examples of the acid addition salt include: inorganic acid salts such as hydrochloride, sulfate, nitrate, phosphate, and perchlorate; organic acid salts such as maleate, fumarate, tartrate, citrate, ascorbate, and trifluoroacetate; and sulfonates such as methanesulfonate, isothiocyanate, benzenesulfonate, and p-toluenesulfonate.

Examples of the base addition salt include: alkali metal salts such as sodium salt and potassium salt; alkali-earth metal salts such as calcium salt and magnesium salt; and organic amine salts such as ammonium salt, trimethylamine salt, triethylamine salt, dicyclohexylamine salt, ethanolamine salt, diethanolamine salt, triethanolamine salt, procaine salt, and N,N′-dibenzylethylenediamine salt.

The following discloses derivatives of the present invention in more detail with reference to specific examples of various symbols used in the formula (I).

R¹ represents a hydrogen atom, halogen, C₁₋₆alkyl, haloC₁₋₆alkyl, C₁₋₆alkyloxy, or haloC₁₋₆alkyloxy.

Specific examples of R¹ include: a hydrogen atom; halogens such as fluoro, chloro, bromo, and iodo; C₁₋₆alkyls such as methyl, ethyl, n-propyl, isopropyl, n-butyl, and t-butyl; haloC₁₋₆alkyls such as chloromethyl, trichloromethyl, fluoromethyl, trifluoromethyl, chloroethyl, and fluoroethyl; C₁₋₆alkyloxys such as methoxy, ethoxy, n-propyloxy, and isopropyloxy; and haloC₁₋₆alkyloxys such as chloromethoxy, trichloromethoxy, fluoromethoxy, trifluoromethoxy, fluoroethoxy, and fluoropropyloxy. Preferably, hydrogen atom is recommended.

R² represents C₁₋₆alkyl or haloC₁₋₆alkyl.

Specific examples of R² include: C₁₋₆alkyls such as methyl, ethyl, n-propyl, isopropyl, n-butyl, and t-butyl; and haloC₁₋₆alkyls such as chloromethyl, trichloromethyl, fluoromethyl, trifluoromethyl, chloroethyl, and fluoroethyl. Preferably, C₁₋₆alkyl is recommended.

R³ represents a hydrogen atom, C₁₋₆alkyl, or haloC₁₋₆alkyl.

Specific examples of R³ include: a hydrogen atom; C₁₋₆alkyls such as methyl, ethyl, n-propyl, isopropyl, n-butyl, and t-butyl; and haloC₁₋₆alkyls such as chloromethyl, trichloromethyl, fluoromethyl, trifluoromethyl, chloroethyl, and fluoroethyl. Preferably, C₁₋₆alkyl or haloC₁₋₆alkyl is recommended. C₁₋₆alkyl is further recommended.

Ar represents a bivalent aryl or a bivalent heteroaryl. The aryl or heteroaryl may be substituted with 1 to 3 substituents selected from the group consisting of halogen, C₁₋₆alkyl, haloC₁₋₆alkyl, C₁₋₆alkyloxy, haloC₁₋₆alkyloxy, hydroxy, amino, monoC₁₋₆alkylamino, diC₁₋₆alkylamino, C₁₋₆alkyloxycarbonyl, C₁₋₆alkyloxycarbonylamino, C₁₋₆alkyloxycarbonyl(C₁₋₆alkyl)amino, C₁₋₆alkylcarbonyl, C₁₋₆alkylcarbonyloxy, C₁₋₆alkylcarbonylamino, C₁₋₆alkylcarbonyl(C₁₋₆alkyl)amino, carbamoyl, monoC₁₋₆alkylcarbamoyl, diC₁₋₆alkylcarbamoyl, carbamoylamino, monoC₁₋₆alkylcarbamoylamino, diC₁₋₆alkylcarbamoylamino, monoC₁₋₆alkylcarbamoyl(C₁₋₆alkyl)amino, diC₁₋₆alkylcarbamoyl(C₁₋₆alkyl)amino, carbamoyloxy, monoC₁₋₆alkylcarbamoyloxy, diC₁₋₆alkylcarbamoyloxy, C₁₋₆alkylsulfonyl, C₁₋₆alkylsulfonylamino, C₁₋₆alkylsulfonyl(C₁₋₆alkyl)amino, sulfamoyl, monoC₁₋₆alkylsulfamoyl, diC₁₋₆alkylsulfamoyl, sulfamoylamino, monoC₁₋₆alkylsulfamoylamino, diC₁₋₆alkylsulfamoylamino, monoC₁₋₆alkylsulfamoyl(C₁₋₆alkyl)amino, and diC₁₋₆alkylsulfamoyl(C₁₋₆alkyl)amino.

Examples of aryl-derived rings for Ar include benzene and naphthalene. Preferably, benzene is recommended.

Examples of heteroaryl-derived rings for Ar include pyridine, pyrazine, pyrimidine, pyridazine, thiophene, thiazole, oxazole, thiadiazole, oxadiazole, furan, and pyrrole. Preferably, pyridine and thiazole are recommended.

Preferably, Ar is either unsubstituted or optionally substituted with a substituent, for which, for example, halogens such as fluoro and chloro; C₁₋₆alkyls such as methyl, ethyl, and isopropyl; haloC₁₋₆alkyls such as fluoromethyl and trifluoromethyl; C₁₋₆alkyloxys such as methoxy, ethoxy, and isopropyloxy; and haloC₁₋₆alkyloxys such as fluoromethoxy and trifluoromethoxy are recommended. It is particularly recommended that Ar be either unsubstituted or optionally substituted with a halogen (especially, fluoro).

Specific examples of Ar include the following:

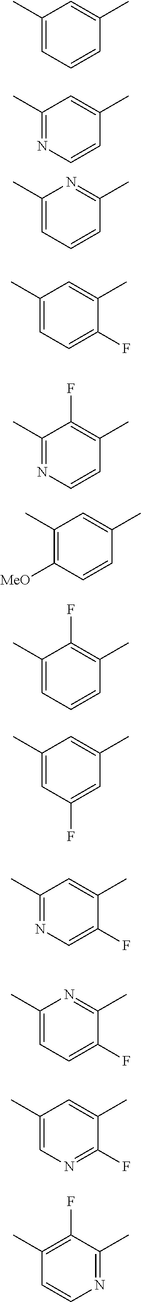

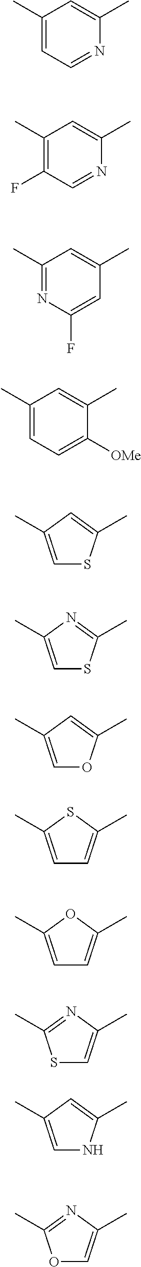

Preferably, the following are recommended.

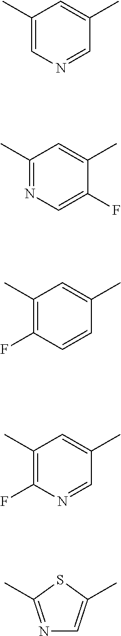

Specific examples of compounds of the present invention include the following.

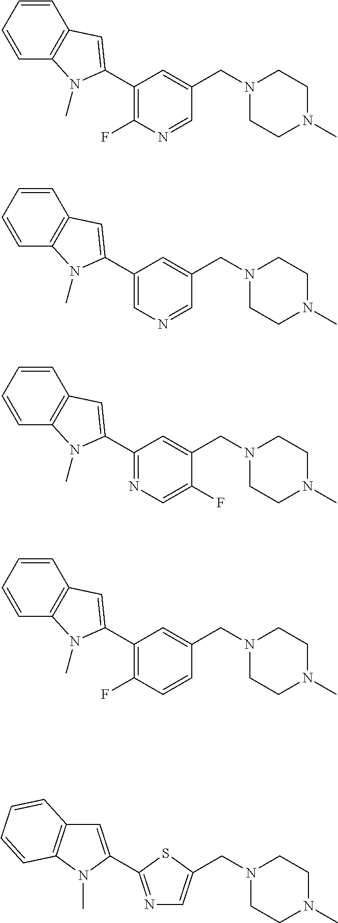

### Producing Methods of the Compound of the Formula (I)

A compound of the present invention can be produced by the methods below.

### Producing Method 1

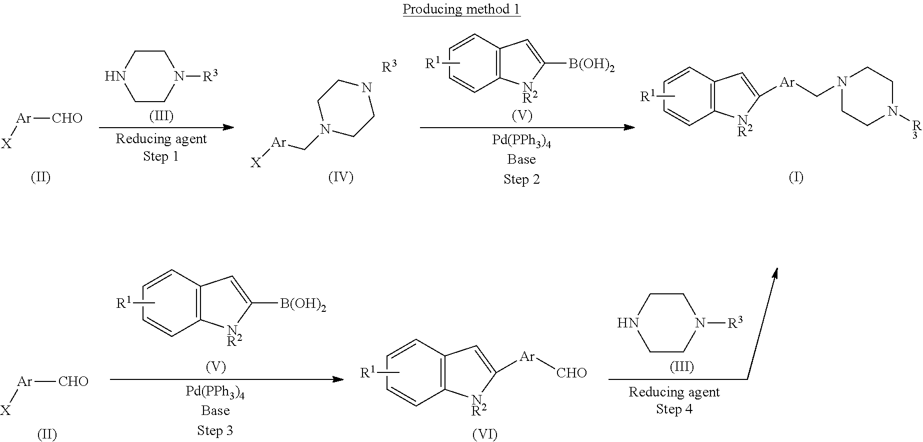

In the formulae, X represents chloro, bromo, or iodo. The other symbols are as defined above.

### Step 1

A compound of a formula (IV) is obtained by the reductive alkylation between a compound of a formula (II) and a compound of a formula (III) in an organic solvent in the presence of a reducing agent.

The compound of the formula (III) is used in an amount of, for example, 1.0 to 2.0 moles, preferably 1.0 to 1.5 moles per mole of the compound of the formula (II).

Examples of the reducing agent include sodium cyanoborohydride, sodium triacetoxyborohydride, sodium borohydride, and triethylsilane, of which sodium cyanoborohydride and sodium triacetoxyborohydride are preferable.

The amount of reducing agent used is generally 1 to 20 equivalents, preferably 1 to 5 equivalents per equivalent of the compound of the formula (III).

The organic solvent is not particularly limited, as long as it does not interfere with the reaction. Examples include methanol, ethanol, chloroform, methylene chloride, 1,2-dichloroethane, tetrahydrofuran (hereinafter, “THF”), and 1,4-dioxane, of which methanol, chloroform, methylene chloride, and 1,2-dichloroethane are preferable.

The reaction may be performed in the presence of a base. Examples of the base include triethylamine, trimethylamine, N,N-diisopropylethylamine, N-methylmorpholine, N-methylpyrrolidine, and N-methylpiperidine.

The amount of base used is generally 0 to 5 equivalents, preferably 0 to 2 equivalents per equivalent of the compound of the formula (III).

As required, additives, such as zinc chloride, acetic acid, and trifluoroacetic acid (hereinafter, “TFA”), may be added to the reaction system. For example, in the case of zinc chloride, approximately 0.01 to 2 moles may be added per mole of the reducing agent. In the case of acetic acid or TFA, the additive may be added in a molar excess with respect to the reducing agent.

The reaction temperature is, for example, 0 to 100° C., preferably 0 to 50° C., and the reaction generally completes in 10 minutes to 48 hours, preferably 10 minutes to 24 hours.

Examples of the compound represented by the formula (II) include the following.

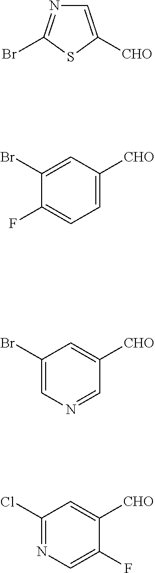

Examples of the compound represented by the formula (III) include the following.

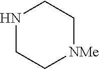

### Step 2

The compound of the formula (I) is obtained by the reaction of a compound of a formula (IV) with a compound of a formula (V) in an organic solvent in the presence of tetrakis(triphenylphosphine)palladium and a base.

Examples of the base include sodium carbonate, potassium carbonate, sodium bicarbonate, sodium hydroxide, and potassium hydroxide.

The compound of the formula (V) is used in an amount of, for example, 1.0 to 2.0 moles, preferably 1.2 moles per mole of the compound of the formula (N).

The amount of tetrakis(triphenylphosphine)palladium used is, for example, 0.05 to 0.10 moles, preferably 0.1 moles per mole of the compound of the formula (N). The amount of base used is, for example, 1.5 to 5.0 moles, preferably 3 to 4 moles per mole of the compound of the formula (IV).

Examples of the organic solvent include a toluene-ethanol mixed solvent, and dimethoxyethane (hereinafter, “DME”).

The reaction temperature is, for example, at room temperature, or under the reflux of the solvent used. The reaction generally completes in 8 to 72 hours.

Examples of the compound represented by the formula (V) include the following.

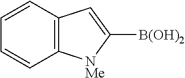

### Steps 3 and 4

Steps 3 and 4 are the reaction of which order of Steps 1 and 2 is changed, and the same reaction conditions and reagents used in Steps 1 and 2 can be used.

### Producing Method 2

Producing method 2 is a method for producing the compound of the formula (I) in which Ar is a pyridine ring, specifically, a compound of a formula (I′).

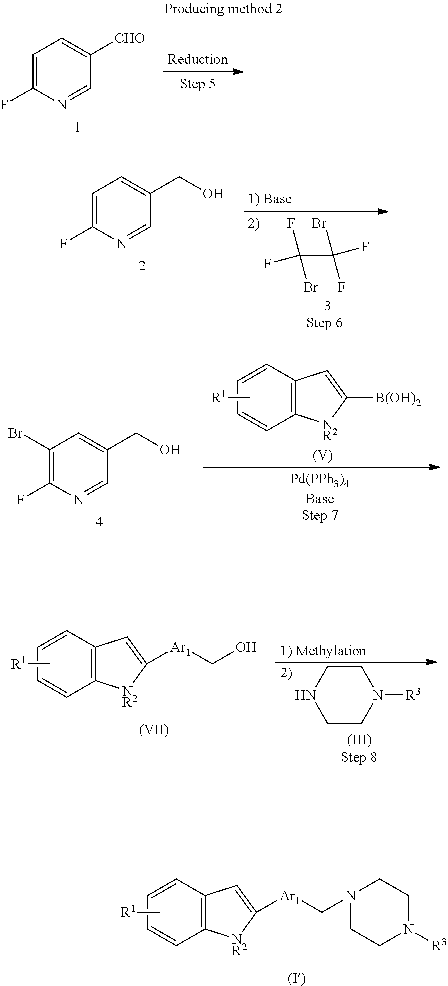

In the formulae, Ar₁ represents 2-fluoropyridine-3,5-diyl. The other symbols are as defined above.

### Step 5

Compound 1 is reduced in an organic solvent to give Compound 2.

The reducing agent may be, for example, sodium borohydride, and the reaction is generally performed in an organic solvent such as methanol and ethanol. The reaction conditions may be those known to those skilled in the art.

### Step 6

Compound 2, after treatment with a base in an organic solvent, is reacted with 1,2-dibromotetrafluoroethane 3 to give Compound 4.

Examples of the base include n-butyllithium and t-butyllithium. Tetramethylethylenediamine may be further added.

The amount of base used is, for example, 1.0 to 2.0 moles per mole of Compound 2.

The base treatment is performed in a temperature range of, for example, −30 to −78° C., generally for 1 to 4 hours.

Examples of the organic solvent include methylene chloride, THF, and N,N-dimethylformamide (hereinafter,“DMF”).

Compound 3 is added to the above reaction mixture to further allow reaction for 30 minutes to 1 hour at 0° C. to 60° C. to give Compound 4.

The amount of Compound 3 used is, for example, 1.0 to 4.0 moles, preferably 2.0 to 3.0 moles per mole of Compound 2.

### Step 7

Compound 4 is reacted with the compound of the formula (V) as in Step 2 to give a compound of a formula (VII).

### Step 8

The compound of the formula (VII) is mesylated using a known method (for example, methanesulfonylchloride/base reaction), and the resulting mesylated product is condensed with the compound of the formula (III) in an organic solvent in the presence of a base to give the compound of the formula (I′).

The condensation of the mesylated product and the compound of the formula (III) can be performed using known methods. For example, the reaction may be performed in a temperature range of from room temperature to about 60° C. in an organic solvent such as methylene chloride and THF, in the presence of a base such as cesium carbonate, triethylamine, and diisopropylethylamine.

In Producing Method 2, the reaction can be effected in the same manner using, for example, the compounds of the formulae below instead of Compound 1.

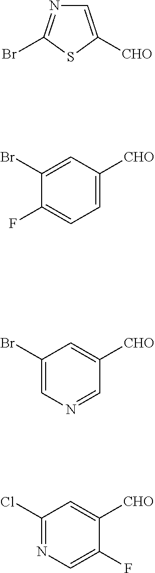

A corresponding compound of the formula (I) can also be prepared in this manner.

In the foregoing producing methods, when the reactants of the reaction include groups, such as amino, hydroxy, carboxyl, oxo, and carbonyl, not involved in the reaction, the reactions of the producing method may be performed after appropriately protecting such an amino, hydroxy, carboxyl, oxo, or carbonyl group with an amino protective group, a hydroxy protective group, a carboxyl protective group, or an oxo or carbonyl protective group, which can be removed after the reaction.

The method of introducing and removing the protective group, though it depends on the type of the protective group and the stability and other properties of the target compound, can be performed, for example, by solvolysis using an acid or a base, according to the method described in Protective Groups in Organic Synthesis, T. W. Greene, John Wiley & Sons, 1981, or methods analogous thereto; specifically, by methods employing, for example, 0.01 moles to a large excess of an acid, preferably such as trifluoroacetic acid, formic acid, or hydrochloric acid, or an equimolar amount to a large excess of a base, preferably such as potassium hydroxide or calcium hydroxide, or by methods employing chemical reduction using compounds such as a metal hydride complex, or catalytic reduction using catalysts such as palladium-carbon catalyst, and Raney nickel catalyst.

The protective group of the amino is not particularly limited, as long as it functions as intended. Examples include: aralkyls such as benzyl, p-methoxybenzyl, and trityl; lower alkanoyls such as acetyl and pivaloyl; benzoyl; lower alkyloxycarbonyls such as methoxycarbonyl, ethoxycarbonyl, and tert-butoxycarbonyl; alkyloxycarbonyls such as benzyloxycarbonyl; lower alkylsilyls such as trimethylsilyl, and tert-butyldimethylsilyl; tetrahydropyranyl; trimethylsilylethoxymethyl; lower alkylsulfonyls such as methylsulfonyl, and ethylsulfonyl; and arylsulfonyls such as benzenesulfonyl, and toluenesulfonyl. Particularly preferable examples include acetyl, benzoyl, tert-butoxycarbonyl, trimethylsilylethoxymethyl, and methylsulfonyl.

The protective group of the hydroxy is not particularly limited, as long as it functions as intended. Examples include: lower alkyls such as methyl, ethyl, and tert-butyl; lower alkylsilyls such as trimethylsilyl, and tert-butyldimethylsilyl; lower alkyloxymethyls such as methoxymethyl, and 2-methoxyethoxymethyl; tetrahydropyranyl; trimethylsilylethoxymethyl; aralkyls such as benzyl, p-methoxybenzyl, and 2,3-dimethoxybenzyl; and acyls such as acetyl. Particularly preferable examples include methyl, methoxymethyl, tetrahydropyranyl, trityl, trimethylsilylethoxymethyl, tert-butyldimethylsilyl, and acetyl.

The protective group of the carboxyl is not particularly limited, as long as it functions as intended. Examples include: lower alkyls such as methyl, ethyl, and tert-butyl; halo lower alkyls such as 2,2,2-trichioroethyl; lower alkenyls such as a 2-propenyl group; and aralkyls such as benzyl, p-methoxybenzyl, benzhydryl, and trityl. Particularly preferable examples include methyl, ethyl, tert-butyl, 2-propenyl, benzyl, p-methoxybenzyl, and benzhydryl.

The protective group of the carbonyl is not particularly limited, as long as it functions as intended. Examples include acetals and ketals such as ethylene ketal, dimethyl ketal, and S,S-dimethyl ketal.

The compound of the formula (I) obtained as above can easily be isolated and purified using common separation means, for example, such as solvent extraction, recrystallization, column chromatography, and preparative thin-layer chromatography.

### Pharmaceutical Composition Containing the Compound of the Formula (I)

The compound of the formula (I) can be orally or parenterally administered, and can be prepared into a suitable administration form expected to be useful for the prevention or treatment of, for example, cardiovascular disease such as hypertension, arterial sclerosis, renal disease, heart disease, and angiospasm; bulimia; and metabolic disease such as obesity, diabetes mellitus, abnormal hormone secretion, hypercholesteremia, hyperlipidemia, gout, and fatty liver. The compound of the formula (I) can therefore be provided as a preventive or remedy for, for example, pain, abnormal circadian rhythms, atherosclerosis, obesity-related gastroesophageal reflux, obesity-hypoventilation syndrome (Pickwickian syndrome), hypertriglyceridemia, low HDL cholesteremia, cardiovascular disease (for example, such as coronary artery heart disease (CHD), cerebrovascular disease, stroke, peripheral vascular disease, and sudden death), pain, osteoporosis-related disease, lower back pain, and anesthetic hypersensitivity, and particularly for obesity.

In clinical use of a compound of the present invention, the compound may generally be administered after being prepared into various dosage forms with a pharmaceutically acceptable carrier in a manner suitable for the administration form. In this case, a variety of carriers commonly used in the field of pharmaceuticals can be used. Specific examples include gelatin, lactose, sucrose, titanium oxide, starch, microcrystalline cellulose, hydroxypropylmethyl cellulose, carboxymethyl cellulose, corn starch, microcrystalline wax, white vaseline, magnesium aluminometasilicate, anhydrous calcium phosphate, citric acid, trisodium citrate, hydroxypropyl cellulose, sorbitol, sorbitan fatty acid ester, polysorbate, sucrose fatty acid ester, polyoxyethylene, hydrogenated castor oil, polyvinylpyrrolidone, magnesium stearate, light anhydrous silicic acid, talc, vegetable oil, benzyl alcohol, gum arabic, propylene glycol, polyalkylene glycol, cyclodextrin, and hydroxypropylcyclodextrin.

Examples of the dosage forms prepared with the carrier include: solid preparations such as a tablet, a capsule, a granule, a powder, and a suppository; and liquid preparations such as a syrup, an elixir, and an injection. These can be prepared according to methods commonly used in the field of pharmaceuticals. The liquid preparation may be prepared by being dissolved or suspended in water or other suitable media before use. Specifically, the injection may be prepared by being dissolved or suspended in physiological saline or glucose solution as required, and may further include buffer or preservative.

The preparation may contain a compound of the present invention in a proportion of 1 to 99.9 weight %, preferably 1 to 60 weight % based on its pharmaceutical composition. The preparation may further contain other therapeutically effective compounds.

Specifically, the present invention provides a pharmaceutical composition that contains a pharmaceutically acceptable carrier, and a therapeutically effective amount of a compound of the present invention or a pharmaceutically acceptable salt thereof.

The therapeutically effective amount, as that term is used herein, means the amount of medicament that induces biological or medical events in tissues, systems, animals, or humans, as determined by researchers, veterinarians, physicians or other clinicians.

Specifically, in using a compound of the present invention for the prevention or treatment of the diseases such as above, the dose and dosing frequency can vary depending on such factors as the sex, age, and body weight of a patient, the level of symptoms, and the type and range of intended effect. Generally, in oral administration, the dosage generally may be 0.001 to 50 mg per kilogram body weight per day, given as a single dose or in multiple portions. Preferably, the dosage is about 0.01 to about 25 mg/kg per day, more preferably about 0.05 to about 10 mg/kg per day.

A compound of the present invention can be used for combination therapy with drugs (hereinafter, “co-drugs”) that are effective for diseases such as hypertension, obesity-related hypertension, hypertension-related disease, cardiac hypertrophy, left ventricular hypertrophy, metabolic disease, obesity, and obesity-related disease. The drug can be simultaneously, separately, or successively administered for the prevention or treatment of the disease. When using a compound of the present invention simultaneously with one or more co-drugs, they may be prepared as a pharmaceutical composition of a single administration form. However, in combination therapy, the co-drug and a composition containing a compound of the present invention may be simultaneously, separately, or successively administered to a subject in different packages. The packages may be given with a time lag.

The dose of the co-drug may be in accordance with that used in the clinic, and may be appropriately selected according to such factors as the subject, administration route, disease, and combination. The form of administration of the co-drug is not particularly limited, as long as the co-drug has been combined with a compound of the present invention at the time of administration.

Examples of the administration form include: (1) administration of a single preparation simultaneously prepared from a compound of the present invention and the co-drug, (2) simultaneous administration of two preparations separately prepared from a compound of the present invention and the co-drug, via the same administration route, (3) separate administration of two preparations prepared from a compound of the present invention and the co-drug, via the same administration route, (4) simultaneous administration of two preparations separately prepared from a compound of the present invention and the co-drug, via different administration routes, and (5) time-lagged administration of two preparations separately prepared from a compound of the present invention and the co-drug, via different administration routes (for example, the administration of a compound of the present invention and the co-drug in this order, and vice versa). The proportions of a compound of the present invention and the co-drug can be appropriately selected according to such factors as the subject, administration route, and disease.

Examples of the co-drug usable in the present invention include remedies for diabetes mellitus, hyperlipidemia, and hypertension, and anti-obesity drugs. Two or more kinds of co-drugs may be used in combination in appropriate proportions.

The usefulness of a compound according to the present invention as a medicament is demonstrated by, for example, the following pharmacological test example 1.

### Pharmacological Test Example 1 (QRFP43 Binding Inhibition Test)

The cDNA sequence [Accession No. NM_{—}198179] that codes for human QRFP receptor (GPR103) was cloned into an expression vector pEF1V5-HisB (Invitrogen). The expression vector so prepared was transfected into NFAT β-Lactamase CHO-K1 host cells (Aurora) to obtain QRFP receptor (GPR103) expressing cells, using the cationic lipid method [see Proceedings of the National Academy of Sciences of the United States of America, Vol. 84, p. 7413 (1987)].

The membrane specimen prepared from the QRFP receptor (GPR103) expressing cells was incubated at 25° C. for 1 hour with a test compound and 20,000 cpm [¹²⁵I ] QRFP43 (PerkinElmer, Inc.) in an assay buffer (50 mM Tris-HCl, 1 mM EDTA, and 0.1% BSA, pH 7.4), followed by filtration using a glass filter GF/C. After washing with a 50 mM Tris-HCl (containing 2 mM EDTA, 10 mM MgCl2, and 0.04% Tween-20) buffer at pH 7.4, the radioactivity on the glass filter was determined using a gamma counter. Non-specific binding was measured in the presence of 1 μM peptide QRFP43, and 50% inhibition concentration (IC₅₀ value) of the test compound for the specific [¹²⁵I] QRFP43 binding was determined [see Endocrinology, Vol. 131, p. 2090 (1992)]. The results are shown in the table below.

TABLE 1

| No | Structural formula | IC50(nM) |
|---|---|---|
| 1 | 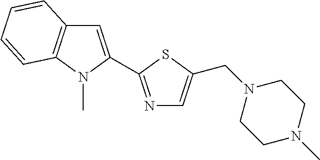 | 49.75 |
| 2 | 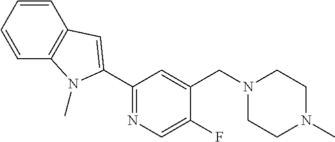 | 33.08 |
| 3 | 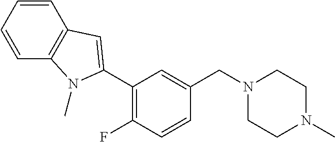 | 48.27 |
| 4 | 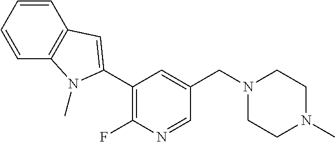 | 52.52 |
| 5 | 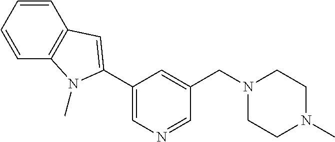 | 111.4 |

As can be seen from the results, the compounds of the present invention strongly inhibited the [¹²⁵I] QRFP43 binding to the QRFP43 receptor (GPR103).

As demonstrated above, a compound according to the present invention is expected to be useful for the prevention or treatment of a variety of diseases involving QRFP43 or 26RFa, including, for example, cardiovascular disease such as hypertension, arterial sclerosis, renal disease, heart disease, and angiospasm; bulimia; and metabolic disease such as obesity, diabetes mellitus, abnormal hormone secretion, hypercholesteremia, hyperlipidemia, gout, and fatty liver (see, for example, Non-Patent Document 1, Patent Document 1 and Patent Document 2). A compound of the present invention can be provided as a preventive or remedy for, for example, pain, abnormal circadian rhythms, atherosclerosis, obesity-related gastroesophageal reflux, obesity-hypoventilation syndrome (Pickwickian syndrome), hypertriglyceridemia, low HDL cholesteremia, cardiovascular disease (for example, such as coronary artery heart disease (CHD), cerebrovascular disease, stroke, peripheral vascular disease, and sudden death), pain, osteoporosis-related disease, lower back pain, and anesthetic hypersensitivity, and particularly for obesity.

### EXAMPLES

The present invention is described below more specifically based on examples. It should be noted, however, that the invention is in no way limited by the description of the following examples. As the column silica gel, Wakogel™ C-200 (Wako Pure Chemical Industries, Ltd.) was used. As the packed silica gel column, the disposable column (Si series, NH series; Moritex Corporation), FLASH+™ cartridge, KP-Sil or FPNH, FLASH 12+M, FLASH 25+S, FLASH 25+M, FLASH 40+M, or the like (Biotage Japan), TC-C18 (Agilent), or Extend-C18(Zorbax) was used. For preparative thin-layer chromatography, Kieselgel 60F254 (Merck) was used. Mass spectra were measured using Quattro II (Micromass). For the ¹HNMR measurement, JNM-AL400 (JEOL) or MERCURY vx400 (VARIAN), and ^{UNITY}INOVA 400 (VARIAN) were used. ZQ 2000 (Waters) was used for the mass spectral measurement.

### Production Example 1

### 2-(1-Methyl-1H-indol-2-yl)-1,3-thiazole-5-carbaldehyde

A dimethoxyethane solution (15 mL) of commercially available (1-methyl-1H-indol-2-yl)boronic acid (328 mg), commercially available 2-bromo-1,3-thiazole-5-carbaldehyde (300 mg), tetrakis(triphenylphosphine)palladium(0) (181 mg), and 1 M sodium carbonate aqueous solution (4.7 mL) was stirred overnight at room temperature in a nitrogen atmosphere. The dimethoxyethane was distilled off under reduced pressure, and the resulting residue was extracted with chloroform, and dried over anhydrous magnesium sulfate. The solvent was distilled off under reduced pressure, and the resulting residue was purified by silica gel column chromatography (hexane:ethyl acetate=3:2) to give the title compound (160 mg) as a pale yellow solid.

### Production Example 2

### 1-[(2-Chloro-5-fluoropyridin-4-yl)methyl]-4-methylpiperazine

1-Methylpiperazine (0.42 mL), and sodium triacetoxyborohydride (811 mg) were added to a 1% acetic acid-chloroform solution (10 mL) of commercially available 2-chloro-5-fluoroisonicotinaldehyde (555 mg), and the mixture was stirred at room temperature for 5 hours. After addition of a 1 N sodium hydroxide aqueous solution, the reaction mixture was extracted with chloroform, and dried over anhydrous sodium sulfate. The solvent was evaporated under reduced pressure to give a crude product of the title compound (850 mg).

### Production Example 3

### 1-(3-Bromo-4-fluorobenzyl)-4-methylpiperazine

The title compound (1.1 g) was obtained as a colorless oily substance according to the procedure of Example 1, using commercially available 3-bromo-4-fluorobenzaldehyde (1.0 g) and 1-methylpiperazine (1.3 mL).

### Production Example 4-1

### (6-Fluoropyridin-3-yl)methanol

Sodium borohydride (2.4 g) was gradually added to a methanol solution (100 mL) of commercially available 6-fluoronicotinaldehyde (7.8 g) under ice-cooled conditions. The mixture was stirred at the same temperature for 1 hour, and water was added after removing the methanol under reduced pressure. After extracting the reaction mixture with ethyl acetate, the organic layer was washed with water and saturated brine, and dried over anhydrous sodium sulfate. The solvent was evaporated under reduced pressure, and the residue was purified by silica gel column chromatography (hexane:ethyl acetate=2:1) to give the title compound (4.9 g) as a colorless solid.

### Production Example 4-2

### (5-Bromo-6-fluoropyridin-3-yl)methanol

A THF solution (200 mL) of the compound (4.9 g) obtained in Production Example 4-1 and N,N,N′,N′-tetramethylethylenediamine (14.4 mL) was cooled to −78° C., and a hexane solution (25 mL) of 1.63 M n-butyllithium, and a heptane solution (36.2 mL) of 1.58 M t-butyllithium were successively added dropwise. The reaction mixture was stirred at −78° C. for 1 hour, gradually raised to −35° C. to −30° C., and further stirred at the same temperature for 2 hours. After cooling to −78° C., 1,2-dibromotetrafluoroethane (13 mL) was gradually added dropwise. The reaction mixture was stirred at −78° C. for 5 minutes, gradually raised to room temperature, and further stirred at room temperature for 30 minutes. After treatment with a saturated ammonium chloride solution and extraction with ethyl acetate, the organic layer was washed with water and saturated brine, and dried over anhydrous sodium sulfate. The solvent was evaporated under reduced pressure, and the residue was purified by silica gel column chromatography (hexane:ethyl acetate=3:1) to give the title compound (1.8 g) as a yellow solid.

### Production Example 4-3

### [6-Fluoro-5-(1-methyl-1H-indol-2-yl)pyridin-3-]methanol

The title compound (1.1 g) was obtained as a yellow solid according to the procedure of Example 2, using the compound (1.8 g) obtained in Production Example 4-2, commercially available (1-methyl-1H-indol-2-yl)boronic acid (1.6 g), tetrakis(triphenylphosphine)palladium(0) (840 mg), and 2 M sodium carbonate aqueous solution (20 mL).

### Production Example 5

### 1-[(5-Bromopyridin-3-yl]methyl]-4-methylpiperazine

The title compound (942 mg) was obtained as a brown oily substance according to the procedure of Example 1, using commercially available 5-bromo-3-pyridinecarboxaldehyde (1.0 g) and 1-methylpiperazine (1.3 mL).

### Example 1

### 1-Methyl-2-{5-[(4-methylpiperazin-1-yl)methyl]-1,3-thiazol-2-yl}-1H-indole

Zinc chloride (9 mg) and sodium cyanoborohydride (124 mg) were added to a methanol solution (3 mL) of the compound (160 mg) obtained in Production Example 1 and 1-methylpiperazine (0.11 mL), and the mixture was stirred overnight at room temperature. After addition of a saturated sodium bicarbonate aqueous solution, the methanol was distilled off under reduced pressure. The resulting residue was extracted with chloroform, and dried over anhydrous magnesium sulfate. The solvent was distilled off under reduced pressure, and the resulting residue was purified using NH-silica gel column chromatography (hexane:ethyl acetate=3:2) to give the title compound (170 mg) as a pale yellow solid.

¹H-NMR (400 MHz, CDCl₃, δppm): 2.30 (3H, s), 2.53 (8H, m), 3.76 (2H, s), 4.15 (3H, s), 6.95 (1H, s), 7.14 (1H, br t, J=7.4 Hz), 7.29 (1H, m), 7.38 (1H, br d, J=8.2 Hz), 7.63 (2H, m).

ESI-MS Found: m/z 327[M+H]⁺

### Example 2

### 2-{5-Fluoro-4-[(4-methylpiperazin-1-yl)methyl]pyridin-2-yl)}-1-methyl-1H-indole.fumarate

The compound (850 mg) obtained in Production Example 2, commercially available (1-methyl-1H-indol-2-yl)boronic acid (671 mg), and tetrakistriphenylphosphine palladium (403 mg) were added to a mixed solution of toluene (30 mL)-ethanol (15 mL)-2 M sodium carbonate aqueous solution (7 mL). After flushing the reaction system with argon gas, the mixture was stirred at 100° C. over night. After cooling, the reaction mixture was extracted with ethyl acetate. The organic layer was then washed with water and saturated brine, and dried over anhydrous sodium sulfate. The solvent was evaporated under reduced pressure, and the residue was purified by silica gel column chromatography (chloroform:methanol=100:1 to 70:1) to obtain a free form of the title compound (284 mg) as a yellow oily liquid. At room temperature, an ethanol solution (8 mL) of fumaric acid (97 mg) was gradually added to a stirred ethyl acetate solution (40 mL) of the oily liquid obtained as above. After 1-hour stirring at room temperature, the precipitate was filtered off, and thoroughly washed with ethyl acetate. The resulting solid was dried under reduced pressure to give the title compound (250 mg) as a white solid.

1H-NMR (400 MHz, DMSO-d6, δppm): 2.26 (3H, s), 2.40-2.60 (8H, m), 3.67 (2H, s), 4.00 (3H, s), 6.59 (2H, s), 6.92 (1H, s), 7.07-7.11 (1H, m), 7.21-7.25 (1H, m), 7.52 (1H, d, J=8.0 Hz), 7.61 (1H, d, J=8.0 Hz), 7.90 (1H, d, J=6.0 Hz), 8.64 (1H, s).

ESI-MS Found: m/z 339[M+H]⁺

### Example 3

### 2-{2-Fluoro-5-[(4-methylpiperazin-1-yl)methyl]phenyl}-1-methyl-1H-indole

The title compound (59 mg) was obtained as a pale brown oily substance according to the procedure of Production Example 1 (except that the reaction temperature was 90° C., and the reaction time was 3 days), using the compound (91.4 mg) obtained in Production Example 3, commercially available (1-methyl-1H-indol-2-yl)boronic acid (66.8 mg), tetrakis(triphenylphosphine)palladium(0) (36.8 mg), and a 2 M sodium carbonate aqueous solution (0.64 mL).

¹H-NMR (400 MHz, DMSO-d6, δppm): 2.14 (3H, s), 2.36 (8H, m), 3.52 (2H, s), 3.63 (3H, s), 6.57 (1H, s), 7.09 (1H, m), 7.21 (1H, m), 7.34 (1H, m), 7.43 (2H, m), 7.50 (1H, d, J=8.2 Hz), 7.59 (1H, d, J=7.8 Hz).

ESI-MS Found: m/z 338[M+H]⁺

### Example 4

### 2-{(2-Fluoro-5-[(4-methylpiperazin-1-yl)methyl]pyridin-3-yl}-1-methyl-1H-indole.difumarate

Under ice-cooled, triethylamine (0.5 mL) and methanesulfonyl chloride (0.28 mL) were successively added to a THF solution (6 mL) of the compound (613 mg) obtained in Production Example 4-3, and the mixture was stirred at 0° C. for 30 minutes. The precipitated triethylamine hydrochloride was quickly filtered off, and washed with THF. Cesium carbonate (1.95 g) and 1-methylpiperazine (1 mL) were added to the resulting filtrate, which was then stirred overnight at room temperature. The reaction mixture was filtered and washed with THF, and the filtrate was concentrated under reduced pressure. The residue was purified by silica gel column chromatography (chloroform:methanol=100:1 to 60:1) to give a free form of the title compound (800 mg) as a yellow oily liquid. At room temperature, an ethanol solution (12 mL) of fumaric acid (278 mg) was gradually added to a stirred ethyl acetate solution (40 mL) of the oily liquid obtained as above. After 1-hour stirring at room temperature, the precipitate was filtered off, and thoroughly washed with ethyl acetate. The resulting solid was dried under reduced pressure to give the title compound (650 mg) as a white solid.

1H-NMR (400 MHz, DMSO-d6, δppm): 2.31 (3H, s), 2.40-2.65 (8H, m), 3.62 (2H, s), 3.67 (3H, s), 6.60 (4H, s), 6.69 (1H, s), 7.08-7.13 (1H, m), 7.21-7.26 (1H, m), 7.53 (1H, d, J=8.0 Hz), 7.61 (1H, d, J=8.0 Hz), 8.01 (1H, dd, J=9.2, 2.4 Hz), 8.24 (1H, s).

ESI-MS Found: m/z 339[M+H]⁺

### Example 5

### 1-Methyl-2-{5-[(4-methylpiperazin-1-yl)methyl]pyridin-3-yl}-1H-indole

The title compound (86.8 mg) was obtained as a pale brown solid according to the procedure of Production Example 1 (except that the reaction temperature was 90° C., and the reaction time was 3 days), using the compound (101 mg) obtained in Production Example 5, commercially available (1-methyl-1H-indol-2-yl)boronic acid (79 mg), tetrakis(triphenylphosphine)palladium(0) (43.5 mg), and 2 M sodium carbonate aqueous solution (0.75 mL).

¹H-NMR (400 MHz, DMSO-d6, δppm): 2.15 (3H, s), 2.39 (8H, m), 3.60 (2H, s), 3.76 (3H, s), 6.69 (1H, s), 7.09 (1H, m), 7.22 (1H, m), 7.53 (1H, d, J=7.8 Hz), 7.60 (1H, d, J=7.8 Hz), 7.90 (1H, m), 8.54 (1H, d, J=2.0 Hz), 8.71 (1H, d, J=2.0 Hz).

ESI-MS Found: m/z 321[M+H]⁺

### INDUSTRIAL APPLICABILITY

A compound according to the present invention is expected to be useful for the prevention or treatment of a variety of diseases involving QRFP43 or 26RFa, including, for example, cardiovascular disease such as hypertension, arterial sclerosis, renal disease, heart disease, and angiospasm; bulimia; and metabolic disease such as obesity, diabetes mellitus, abnormal hormone secretion, hypercholesteremia, hyperlipidemia, gout, and fatty liver. A compound of the present invention can therefore be provided as a preventive or reemdy for, for example, pain, abnormal circadian rhythms, atherosclerosis, obesity-related gastroesophageal reflux, obesity-hypoventilation syndrome (Pickwickian syndrome), hypertriglyceridemia, low HDL cholesteremia, cardiovascular disease (for example, such as coronary artery heart disease (CHD), cerebrovascular disease, stroke, peripheral vascular disease, and sudden death), pain, osteoporosis-related disease, lower back pain, and anesthetic hypersensitivity.

1. A compound represented by a formula (I) or a pharmaceutically acceptable salt thereof: 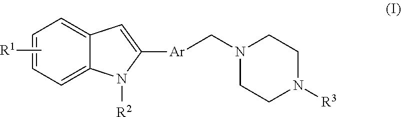
wherein R¹ represents a hydrogen atom, halogen, C₁₋₆alkyl, haloC₁₋₆alkyl, C₁₋₆alkyloxy, or haloC₁₋₆alkyloxy;
R² represents C₁₋₆alkyl or haloC₁₋₆alkyl;
R³ represents a hydrogen atom, C₁₋₆alkyl, or haloC₁₋₆alkyl; and
Ar represents an aryl or heteroaryl, wherein the aryl or the heteroaryl may be substituted with 1 to 3 substituents selected from a group consisting of halogen, C₁₋₆alkyl, haloC₁₋₆alkyl, C₁₋₆alkyloxy, haloC₁₋₆alkyloxy, hydroxy, amino, monoC₁₋₆alkylamino, diC₁₋₆alkylamino, C₁₋₆alkyloxycarbonyl, C₁₋₆alkyloxycarbonylamino, C₁₋₆alkyloxycarbonyl(C₁₋₆alkyl)amino, C₁₋₆alkylcarbonyl, C₁₋₆alkylcarbonyloxy, C₁₋₆alkylcarbonylamino, C₁₋₆alkylcarbonyl(C₁₋₆alkyl)amino, carbamoyl, monoC₁₋₆alkylcarbamoyl, diC₁₋₆alkylcarbamoyl, carbamoylamino, monoC₁₋₆alkylcarbamoylamino, diC₁₋₆alkylcarbamoylamino, monoC₁₋₆alkylcarbamoyl(C₁₋₆alkyl)amino, diC₁₋₆alkylcarbamoyl(C₁₋₆alkyl)amino, carbamoyloxy, monoC₁₋₆alkylcarbamoyloxy, diC₁₋₆alkylcarbamoyloxy, C₁₋₆alkylsulfonyl, C₁₋₆alkylsulfonylamino, C₁₋₆alkylsulfonyl(C₁₋₆alkyl)amino, sulfamoyl, monoC₁₋₆alkylsulfamoyl, diC₁₋₆alkylsulfamoyl, sulfamoylamino, monoC₁₋₆alkylsulfamoylamino, diC₁₋₆alkylsulfamoylamino, monoC₁₋₆alkylsulfamoyl(C₁₋₆alkyl)amino, and diC₁₋₆alkylsulfamoyl(C₁₋₆alkyl)amino.

2. The compound or the pharmaceutically acceptable salt thereof according to claim 1, wherein R¹ is a hydrogen atom.

3. The compound or the pharmaceutically acceptable salt thereof according to claim 1, wherein R² is C₁₋₆alkyl.

4. The compound or the pharmaceutically acceptable salt thereof according to claim 1, wherein R³ is C₁₋₆alkyl.

5. The compound or the pharmaceutically acceptable salt thereof according to claim 1, wherein Ar is an aryl optionally substituted with a halogen.

6. The compound or the pharmaceutically acceptable salt thereof according to claim 1, wherein Ar is a heteroaryl optionally substituted with a halogen.

7. The compound or the pharmaceutically acceptable salt thereof according to claim 5, wherein Ar is a group formed by removal of two hydrogen atoms from a fluorobenzene.

8. The compound or the pharmaceutically acceptable salt thereof according to claim 6, wherein Ar is a group formed by removal of two hydrogen atoms from a pyridine.

9. The compound or the pharmaceutically acceptable salt thereof according to claim 6, wherein Ar is a group formed by removal of two hydrogen atoms from a fluoropyridine.

10. The compound or the pharmaceutically acceptable salt thereof according to claim 6, wherein Ar is a group formed by removal of two hydrogen atoms from a thiazole.

11. The compound or the pharmaceutically acceptable salt thereof according to claim 1, wherein Ar is one of 6-fluorophenylene-1,3-diyl, 3-fluoropyridine-4,6-diyl, 2-fluoropyridine-3,5-diyl, pyridine-3,5-diyl, and thiazole-2,5-diyl.

12. The compound or the pharmaceutically acceptable salt thereof according to claim 1, wherein the compound of the formula (I) is selected from the group consisting of:
1-methyl-2-{5-[(4-methylpiperazin-1-yl)methyl]-1,3-thiazol-2-yl}-1H-indole,
2-{5-fluoro-4-[(4-methylpiperazin-1-yl)methyl]pyridin-2-yl}-1-methyl-1H-indole,
2-{2-fluoro-5-[(4-methylpiperazin-1-yl)methyl]phenyl}-1-methyl-1H-indole,
2-{2-fluoro-5-[(4-methylpiperazin-1-yl)methyl]pyridin-3-yl}-1-methyl-1H-indole, and
1-methyl-2-{5-[(4-methylpiperazin-1-yl)methyl]pyridin-3-yl}-1H-indole.

13. A pharmaceutical composition, which comprises the compound of claim 1, and a pharmaceutically acceptable carrier.

14. (canceled)

15. A method for treating obesity in a subject in need thereof which comprises administering to the subject a therapeutically effective amount of a compound of claim 1, or a pharmaceutically acceptable salt thereof
